(19) United States
(12) Patent Application Publication
(10) Pub. No.: US 2003/0147850 A1
(43) Pub. Date: Aug. 7, 2003

(54) Composition and methods for potentiating therapeutic effects of interferons

(76) Inventors: Igor Anatolievich Pomytkin, Moscow (RU); Pavel Vasilievich Verteletsky, Moscow (RU); Evgeny Nickolayevich Sventytsky, St Petersburg (RU)

Correspondence Address: Lackenbach Siegel Marzullo, Aronson & Greenspan, Penthouse Suite, One Chase Road, Scarsdale, NY, 10583, US

(21) Appl. No.: 10/276,535

(22) PCT Filed: Sep. 27, 2001
(86) PCT No.: PCT/RU01/00389

(51) Int. Cl. A61K 38/21; A61K 31/19

(52) U.S. Cl. 424/85.4; 424/85.6; 514/574

(57) ABSTRACT

This invention relates to compositions and methods for potentiating therapeutic effects of interferons in a mammal. More specifically, the invention relates to compositions comprising interferon and succinic acid or pharmaceutically acceptable salt thereof in amounts sufficient to potentiate therapeutic effects of interferon such as antiviral and antitumor effects. Further, the invention relates to methods for potentiating therapeutic effects of interferon in mammal which methods comprise administering to said mammal, either sequentially in any order or simultaneously, interferon and succinic acid or pharmaceutically acceptable salt thereof in amounts sufficient to potentiate therapeutic effects of interferon such as antiviral and antitumor effects.

### FIELD OF THE INVENTION

The present invention is in the field of medicine. More specifically, this invention relates to compositions and methods for potentiating therapeutic effects of interferons.

### BACKGROUND OF THE INVENTION

Interferons (IFNs) are naturally occurring proteins with antiviral, antiproliferative and immunoregulatory activity. The following definition for interferon has been accepted by international committee assembled to devise a system for the orderly nomenclature of interferons: “To qualify as an interferon a factor must be a protein which exerts virus nonspecific, antiviral activity at least in homologous cells through cellular metabolic processes involving synthesis of both RNA and protein.” J. Interferon Research, 1: pp. vi (1980).

Four distinct classes of interferons are known to exist in humans. Pestka et al., Ann. Rev. Biochem., 56: 727 (1987); Emanuel and Pestka, J. Biol. Chem. 268: 12565 (1993). The three main human interferons are known as IFN-alpha, IFN-beta and IFN-gamma.

The IFN-alpha family represents the predominant class of human IFNs. At least 23 different variants of IFN-alpha are known to date. All known subtypes of IFN-alpha show the same antiviral, antiparasitic, antiproliferative activities although they may differ in relative activities. IFN-alpha is mainly employed as a standard therapy against viral infections such as chronic viral hepatitis caused by hepatitis B and hepatitis C viruses. It is also active against a number of tumors such as hairy cell leukemia, metastasizing renal carcinoma and AIDS-associated angiogenic tumors known as Kaposi sarcomas.

IFN-beta is used for treating multiple sclerosis. IFN-beta in combination with IFN-alpha has been used in the treatment of chronic active hepatitis B. The antiviral activity of IFN-beta is demonstrated also in the treatment of severe childhood viral encephalitis.

IFN-gamma has antiviral and antiparasitic activities and also inhibits the proliferation of a number of normal and transformed cells, but the main biological activity of IFN-gamma appears to be immunomodulatory in contrast to the other interferons, which are mainly antiviral. IFN-gamma has been shown to be effective in the treatment of chronic polyarthritis.

Because human native interferon is expensive to extract, techniques have been developed for preparing recombinant forms of human interferon, for example, human recombinant IFN-alpha2b.

Standardization of interferon potency in International Units (IU) is critical for preclinical research and the clinical development of interferon products as therapeutic agents. Generally, the cytopathic effect inhibition assay is used to standardize interferon products. Rubinstein, Familletti, and Pestka, J. Virol., 37: 755 (1981); Armstrong, “Cytopathic Effect Inhibition Assay for Interferon: Microculture Plate Assay,” in Methods in Enzymology, 78: 381-387 (1981); Familletti, Rubinstein, and Pestka, “A Convenient and Rapid Cytopathic Effect Inhibition Assay for Interferon,” in Methods in Enzymology, 78: 387-394 (1981). In this assay, one unit of interferon is defined as the amount of interferon that reduced virus-induced cytopathic effect by 50 percent, and is calibrated against the international reference standard in International Units.

Therapeutic effectiveness of interferons is frequently diminished under disease states because of an impaired biological response to interferon. This impaired biological response to interferon is known in the art as interferon resistance and well documented for viral diseases such as hepatitis C, AIDS, influenza, and herpes. Goodbourn et al. J.Gen.Virol., 81: 2341-64(2000). Also, the interferon resistance is frequently associated with inflammation and specific cytokine action, especially IL-8. Khabar et al., J.Exp.Med., 186: 1077-85 (1997); Polyak et al., J. Virology, 75: 6095-6106 (2001); Polyak et al., J.Virology. 75: 6209-6211 (2001).

Therapy with interferons is frequently accompanied with adverse events that depend on dosage of the interferon and longevity of the therapy. These adverse events typically include headache, fatigue, rigors, fever, myalgia, arthralgia, and musculoskeletal pain.

Succinic acid is a mammalian (human) metabolite, which plays a role in respiration and energy metabolism. Under a physiological pH, succinic acid exists in form of anion widely known as succinate.

Surprisingly, it has now been found that interferon administered with succinic acid or salt thereof manifest much more pronounced therapeutic effects than interferon administered alone without succinic acid or salt thereof. So, co-administration of interferon and succinate results in more effective protection of cells against virus-induced cytopathology than interferon alone, whereas succinate itself has no effect on the cells protection.

It is an object of the present invention to provide methods for potentiating therapeutic effects of interferon, comprising administering to said mammal interferon and succinic acid or a pharmaceutically acceptable salt thereof.

It is an object of the present invention to provide compositions for potentiating therapeutic effects of interferon, comprising amounts of interferon and succinic acid or a pharmaceutically acceptable salt thereof.

### DETAILED DESCRIPTION OF THE INVENTION

The present invention provides a method for potentiating a therapeutic effect of interferon in a mammal in need thereof, which comprises administering to said mammal an amount of interferon and an effective amount of succinic acid or a pharmaceutically acceptable salt thereof.

The administration of interferon and succinic acid or a pharmaceutically acceptable salt thereof can be sequential in time or simultaneous with the simultaneous method being preferred. For sequential administration, interferon can be administered before or after administration of succinic acid or a pharmaceutically acceptable salt thereof.

Further, the present invention provides a composition for potentiating a therapeutic effect of interferon in a mammal in need thereof, which comprises amounts of interferon and succinic acid or a pharmaceutically acceptable salt thereof, and a pharmaceutically acceptable diluent or carrier.

As used herein, the term “potentiating a therapeutic effect of interferon” means that the effect achieved in a mammal with an amount of interferon when administered with an effective amount of succinic acid or a pharmaceutically acceptable salt thereof is greater than the effect achievable with the same amount of interferon without succinic acid or a pharmaceutically acceptable salt thereof and under otherwise equal conditions.

Because of potentiating a therapeutic effect of interferon, this invention provides particularly advantageous methods of achieving the therapeutic effect with less than therapeutic levels of a interferon. Therefore, in practicing this invention, it is possible to minimize potential adverse effects, which may be associated with larger, therapeutic doses of the interferon and still achieve the therapeutic effect.

A particular advantage of the present invention is that the compositions hereof can comprise amounts of interferon, which are less that those required for compositions containing only interferon without succinic acid or a pharmaceutically acceptable salt thereof. Therefore, compositions comprising reduced amounts of interferon according to this invention afford compositions with reduced side effects, which may be associated with amounts of the interferon necessary to achieve the same therapeutic effects as the compositions of this invention.

Because of potentiating a therapeutic effect of interferon, this invention provides particularly advantageous methods and composition for achieving the therapeutic effect in a mammal with impaired biological response to interferon, a state also is known in the art as interferon resistance. The interferon resistance can be associated with a disease state such as viral disease, inflammation, or action of specific cytokine, especially IL-8. The disease states include, but are not limited to, hepatitis C, AIDS, and influenza.

As discussed above, it is now possible through the practice of this invention to achieve certain desired therapeutic effects using less of an interferon than was heretofore possible. The desired therapeutic effects achievable through the practice of this invention include all known in the art therapeutic effects of interferon. Such effects include, but are not limited to, antiviral, antiproliferative, antitumor, antibacterial, and immunoregulatory action of interferon in mammals. Preferred therapeutic effects achieved according to this invention are antiviral and antitumor effects.

In practicing the methods and compositions of this invention, interferon and succinic acid or a pharmaceutically acceptable salt thereof can be administered in a variety of routes including oral (e.g. through gastrointestinal tract or oral mucosa), intranasal, topical, rectal, by inhalation spray, or parenteral (e.g. subcutaneous, intravenous, or intramuscular injections). When the interferon and succinic acid or a pharmaceutically acceptable salt thereof are administered sequentially, the administration of each can be by the same route or by different routes. Preferably, interferon and succinic acid or a pharmaceutically acceptable salt thereof is administered orally or parenterally.

The compounds of the invention can be administered in a wide variety of different dosage forms, i.e., they may be formulated with various pharmaceutically acceptable carriers or diluents in the form of tablets, sublingual tablets, capsules, lozenges, troches, buccal patches, hard candies, powders, spray, dry spray, aerosols, aqueous solutions, elixirs, syrups, and the like. Such carriers include solid diluents or fillers, sterile aqueous media and various non-toxic organic solvents, etc. Other suitable dosage forms for the compounds of this invention include, but are not limited to, controlled release formulations and devices well known to those who practice in the art.

Pharmaceutical ingredients that can be used in the formulation of the present invention may include, but are not limited to, absorbents, buffering agents (such as phosphate buffer, carbonate buffer, tris buffer, tartrate buffer, borate buffer, acetate buffer, or maleate buffer), colorants, flavorants, solvents and co-solvents, coating agents, direct compression excipients, disintegrants, glidants, lubricants, opaquants, polishing agents, suspending agents, sweetening agents, anti-adherents, binders, and capsule diluents, the ingredients may also include anti-fungal preservatives, antimicrobial preservatives, clarifying agents, emulsifying agents, antioxidants, levigating agents, plasticizers, surfactants, tonicity agents, and viscosity increasing agents.

The present invention is not limited in any way to specific interferon but is applicable to all such interferon now known or subsequently discovered or developed. Nonetheless, a preferred interferon for use in the methods and compositions of this invention is human recombinant interferon-alpha.

The amount of interferon to achieve the desired therapeutic effect is within the skill of those who practice in the art having the benefit of the disclosure herein. Typically, interferon will be present in methods and compositions of the invention in amounts within its normal or less dosage unit and daily regimen ranges as detailed in medical literature. The dosage range will be from about 1IU to about 1×10⁷ IU of interferon per subject per day. Preferably, mammals are administered about 1×10⁶ IU to about 5×10⁶ IU human recombinant interferon-alpha per subject per day.

The amounts of interferon to be employed according to this invention may be varied depending upon the condition being treated, the particular compound, and other clinical factors such as weight and condition of the human or animal and the route of administration.

Any suitable succinic acid or a pharmaceutically acceptable salt thereof may be employed in the present invention. The pharmaceutically acceptable salt of the succinic acid is prepared by known methods from organic and inorganic bases. Such bases include, but are not limited to, nontoxic alkali metal and akaline earth bases, for example, calcium, lithium, sodium, and potassium hydroxide; ammonium hydroxide and nontoxic organic bases, such as triethylamine, butylamine, diethanolamine, and triethanolamine.

Succinic acid or a pharmaceutically acceptable salt thereof will be present in methods and compositions of the invention in amounts sufficient to potentiate the therapeutic effect of interferon. The effective amount of succinic acid or a pharmaceutically acceptable salt thereof will typically be from about 0.1 mg to about 250 mg per kg of body per day. Preferably, mammals are administered with about 0.1 mg to 10 mg succinic acid or a pharmaceutically acceptable salt thereof per kg of body per day.

The amounts of succinic acid or a pharmaceutically acceptable salt thereof to be employed according to this invention may be varied depending upon the condition being treated, the particular compound, and other clinical factors such as weight and condition of the human or animal and the route of administration.

The following examples are presented to demonstrate the invention. The examples are illustrative only and are not intended to limit the scope of the invention in any way.

### EXAMPLE1

This example shows that co-administration of interferon and succinate results in potentiating the antiviral effect of interferon.

Materials. Recombinant human interferon-alpha2b (IFN) and interleukin-8 (IL-8) were from Institute of Highly Pure Biopreparation, St.Petersburg, Russia. Disodium succinate hexahydrate (Succinate) was from Sigma, USA.

Cell cultures. Human cell line L41M (Russian culture collection, Institute of Cytology RAN, St. Petersburg, Russia) was used.

CPE inhibition bioassay. To assess antiviral activity of interferon alone or in a combination with succinic acid or salt thereof the cytopathic effect (CPE) inhibition bioassay was used as described in details. Armstrong, Methods in Enzymol., v.78 (PtA), pp. 381-387 (1981). Briefly, the cells in triplicate cultures were treated with serial dilutions of substances and compositions of the invention for 24 h. The medium was then decanted, and the cultures were exposed to vesicular stomatitis virus (VSV), and incubated for 24 h to permit development of extensive cytopathology in unprotected cultures. The ability of interferon to inhibit virus-induced cytopathic effect was assessed in terms of end-point interferon titer. The interferon end-point titer was taken as reciprocals of the dilution that gave 50% cell protection in each of triplicate culture.

Treatment. Cells were treated by serial dilutions of IFN (starting titer 1×10⁶ IU/ml) as a control, 0.25 mg/ml disodium succinate hexahydrate (Succinate), or serial dilutions IFN (starting titer 1×10⁶ IU/ml) plus 0.25 mg/ml Succinate and followed by VSV exposition as described above. CPE inhibition was assayed. Data on CPE inhibition are presented in Table 1 in fold increase to interferon control of triplicate cultures. Interferon control refers to the original antiviral IFN activity in terms of IFN titers.

TABLE 1
CPE inhibition.

| Treatment | CPE inhibition, fold increase to IFN control |
|---|---|
| IFN | 1 |
| Succinate | 0 |
| IFN plus Succinate | 6 |

Table 1 shows that co-administration of interferon and succinate results in potentiating the antiviral effect of interferon. Actually, the cell protection effect achieved with the amount of interferon when administered with the effective amount of succinate is 6-fold greater than the effect achieved with the same amount of interferon without succinate. Moreover, the desired 50% cell protection is achieved with 6-fold less interferon concentration when interferon is co-administered with succinate as compared to interferon is administered without succinate.

### EXAMPLE 2

This example shows that co-administration of interferon and succinate results in potentiating the antiviral effect of interferon under interferon resistance conditions.

Materials. Materials were as described in example 1 of the invention.

Cell cultures. Human hepatoma cell line HepG2 (Russian culture collection, Institute of Cytology RAN, St. Petersburg, Russia) was used.

CPE inhibition bioassay was used as described in the example 1 of the invention.

Treatment. Cells were pretreated with IL-8 (30 ng/ml) for 24 hours at 37° C. Cells were used then for the CPE inhibition bioassay. Assay was used as described in the example 1 of the invention. Briefly, cells in triplicate cultures were treated by serial dilutions of IFN (starting titer 1×10⁶ IU/ml), 0.25 mg/ml Succinate, or serial dilutions IFN (starting titer 1×10⁶ IU/ml) plus 0.25 mg/ml Succinate and followed by VSV exposition. In control, cells were used without pretreatment with IL-8 and treated with serial dilutions of IFN (starting titer 1×10⁶ IU/ml). CPE inhibition was assayed. Data are presented in Table 2 in percent to IFN control of triplicate cultures. Percent of IFN control (100%) refers to the original antiviral IFN activity in terms of IFN titers in the absence of IL-8 pretreatment.

TABLE 2
CPE inhibition in cells pretreated with IL-8.

| | | CPE inhibition, % of IFN |
|---|---|---|
| Treatment | IL-8 pretreatment | control |
| IFN | − | 100 |
| IFN | + | 17 |
| Succinate | + | 0 |
| IFN plus Succinate | + | 100 |

Table 2 shows that co-administration of interferon and succinate results in potentiating the antiviral effect of interferon under interferon resistance caused by IL-8. Actually, the cell protection effect achieved with an amount of interferon when administered with an effective amount of succinate is greater than the effect achieved with the same amount of interferon without succinate. Moreover, administration of interferon in conjunction with succinate restores interferon response impaired by IL-8 to control level.

### EXAMPLE 3

This example shows that co-administration of interferon and succinate results in potentiating the antitumor effect of interferon.

Materials. Materials were as described in example 1 of the invention.

Treatment. Myeloma-bearing DBA/BALB(F1) male mice of 10- to 12-weeks aged and 20-25 g weight were prepared by i.p. injection with 2×10⁶ myeloma NS/0 tumor cells prepared from a brei of several stock tumors. Then, mice were randomized and treated i.p. with 1×10⁶ IU/kg IFN, 5 mg/kg Succinate, 1×10⁶ IU/kg IFN plus 5 mg/kg Succinate, or saline (Control) on days 3 -7 following tumor implantation. The effect of the treatments was determined by tumor growth delay and survival increase in comparison with control to a day following tumor implantation. Tumor growth data are presented in Table 3 as a tumor mass means ± SD (n=5 in each group). Survival data are presented in Table 4 in percent to starting number of mice in each group (n=16). Statistical significance of the survival results was determined by log rank method. Statements referring to a significant difference indicate a p value of 0.01 or less.

TABLE 3
Tumor growth in myeloma-bearing mice.

Tumor mass, mg

| Treatment | 16^{th} day | 21^{st} day |
|---|---|---|
| Control | 541 ± 56 | 2226 ± 350 |
| IFN | 459 ± 50 | 1110 ± 180 |
| IFN plus Succinate | 330 ± 40 | 580 ± 40*^{t} |

*Differs significantly from interferon
^{t}Differs significantly from control

TABLE 4
Survival of myeloma-bearing mice.

| | Percent Survival | | |
|---|---|---|---|
| Day | Control | IFN | IFN plus Succinate |
| 31 | 100 | 100 | 100 |
| 32 | 88 | 88 | 100 |
| 33 | 50 | 88 | 100 |
| 34 | 50 | 88 | 100 |
| 35 | 38 | 63 | 100 |
| 36 | 38 | 38 | 100 |
| 37 | 38 | 38 | 100 |
| 38 | 38 | 38 | 100 |
| 39 | 38 | 38 | 100 |
| 40 | 38 | 38 | 100 |
| 41 | 25 | 38 | 100*^{t} |

*Differs significantly from interferon
^{t}Differs significantly from control

Tables 3 and 4 show that co-administration of interferon and succinate results in potentiating the effect of interferon on tumor growth delay and survival increase. Actually, the tumor growth delay and survival increase achieved with a dosage of interferon when administered with an effective amount of succinate is greater than the effect achieved with the same dosage of interferon without succinate.

### EXAMPLE 4

This example shows that co-administration of interferon and succinate results in potentiating the antitumor effect of interferon.

Materials. Materials were as described in example 1 of the invention.

Treatment. Friend erythroleukemia-bearing C57BL male mice of 10-to 12-weeks aged and about 25 g of weight were prepared by i.p. injection with 2×10⁶ erythroleukemia tumor cells prepared from a brei of several stock tumors. Then, mice were randomized and treated sublingually with 1×10⁶ IU/mouse IFN, 3 mg/mouse Succinate, 1×10⁶ IU/mouse IFN plus 3 mg/mouse Succinate, or saline (Control) on days 3-7 following tumor implantation. The effect of the treatments was determined by tumor growth delay in comparison with control to 16^{th} day following tumor implantation. Tumor growth data are presented in Table 5 as a tumor mass means ± SD (n=5 in each group). Statements referring to a significant difference indicate a p value of 0.01 or less.

TABLE 5
Tumor growth in erythroleukemia-bearing mice.

| Treatment | Tumor mass, mg |
|---|---|
| Control | 379 ± 27 |
| IFN | 354 ± 16 |
| IFN plus Succinate | 251 ± 29*^{t} |

*Differs significantly from interferon
^{t}Differs significantly from control

Table 5 shows that co-administration of interferon and succinate results in potentiating the effect of interferon on tumor growth delay. Actually, the tumor growth delay achieved with a dosage of interferon when administered with an effective amount of succinate is greater than the effect achieved with the same dosage of interferon without succinate.

### EXAMPLE 5

This example shows that co-administration of interferon and succinate results in potentiating the antitumor effect of interferon.

Materials. Materials were as described in example 1 of the invention.

Treatment. Tumor-bearing DBA/BALB(F1) male mice of 10- to 12-weeks aged and 20-25 g weight were prepared by i.p. injection with 2×10⁶ P388 tumor cells prepared from a brei of several stock tumors. The tumor-bearing mice were randomized and treated daily i.p. with 1×10⁵ IU/kg IFN, 5 mg/kg Succinate, 1×10⁵ IU/kg IFN plus 5 mg/kg Succinate, or saline (Control) with two breakups in treating on days 5-7 and 12-14 following tumor implantation. The effect of the treatments was determined by tumor growth delay in comparison with control to 14^{th} day following tumor implantation. Tumor growth data are presented in Table 6 as a solid tumor mass means ± SD (n=5 in each group). Statements referring to a significant difference indicate a p value of 0.01 or less.

TABLE 6
Tumor growth in P388 tumor-bearing mice.

| Treatment | Tumor mass, mg |
|---|---|
| Control | 3800 ± 400 |
| IFN | 4800 ± 500 |
| IFN plus Succinate | 2600 ± 300*^{t} |

*Differs significantly from interferon
^{t}Differs significantly from control

Table 6 shows that co-administration of interferon and succinate results in potentiating the antitumor effect of interferon. Actually, the effect of tumor growth delay achieved with a dosage of interferon when administered with an effective amount of succinate is greater than the effect achieved with the same dosage of interferon without succinate.

We claim:

1. A method for potentiating a therapeutic effect of interferon in a mammal in need thereof, which comprises administering to said mammal an amount of interferon and an effective amount of succinic acid or a pharmaceutically acceptable salt thereof.

2. The method as claimed in claim 1 wherein the interferon and succinic acid or a pharmaceutically acceptable salt thereof are administered simultaneously.

3. The method as claimed in claim 1 or 2 wherein the effect comprises antiviral effect.

4. The method as claimed in claim 1 or 2 wherein the effect comprises antitumor effect.

5. The method as claimed in any one of claims 1 to 4 wherein the interferon is administered parenterally.

6. The method as claimed in any one of claims 1 to 4 wherein the interferon is administered orally.

7. The method as claimed in any one of claims 1 to 4 wherein the succinic acid or a pharmaceutically acceptable salt thereof is administered parenterally.

8. The method as claimed in any one of claims 1 to 4 wherein the succinic acid or a pharmaceutically acceptable salt thereof is administered orally.

9. The method as claimed in any one of claims 1 to 8 wherein the amount of succinic acid or a pharmaceutically acceptable salt thereof is about 0.1 to 250 mg per kg of body weight of the mammal.

10. The method as claimed in claim 9 wherein the amount of succinic acid or a pharmaceutically acceptable salt thereof is about 0.1 to 10 mg per kg of body weight of the mammal.

11. The method as claimed in any one of claims 1 to 10 wherein the amount of interferon is about 1 IU to about 1×10⁷ IU per mammal per day.

12. The method as claimed in claim 11 wherein the amount of interferon is about 1×10⁶ IU to about 5×10⁵ IU per mammal per day.

13. The method as claimed in any one of claims 1 to 12 wherein the interferon is human recombinant interferon-alpha.

14. A composition for potentiating a therapeutic effect of interferon in a mammal in need thereof, which comprises amounts of:
(a) interferon, and
(b) succinic acid or a pharmaceutically acceptable salt thereof, and
(c) a pharmaceutically acceptable diluent or carrier.

15. The composition as claimed in claim 14 wherein the effect comprises antiviral effect.

16. The composition as claimed in claim 14 wherein the effect comprises antitumor effect.

17. The composition as claimed in any one of claims 14 to 16 wherein the amount of the interferon is about 1 IU to 1×10⁷ IU.

18. The composition as claimed in claim 17 wherein the amount of the interferon is about 1×10⁶IU to 5×10⁶IU.

19. The composition as claimed in any one of claims 14 to 18 wherein the amount of succinic acid or a pharmaceutically acceptable salt thereof is about 0.1 to 250 mg per kg of body weight of the mammal.

20. The composition as claimed in claim 19 wherein the amount of succinic acid or a pharmaceutically acceptable salt thereof is about 0.1 to 10 mg per kg of body weight of the mammal.

21. The composition as claimed in any one of claims 14 to 20 wherein the interferon is human recombinant interferon-alpha.
